(19) United States
(12) Patent Application Publication
(10) Pub. No.: US 2025/0120984 A1
(43) Pub. Date: Apr. 17, 2025

(54) PREDNISONE ENTERIC-COATED PREPARATION AND PREPARATION METHOD THEREOF

(71) Applicant: WISDRUG INNOVATIVE DRUG RESEARCH (BEIJING) CO.. LTD, Beijing (CN)

(72) Inventors: Sophia ZHOU, Beijing (CN); Leo LEE, Beijing (CN); Clark LEE, Beijing (CN); Kent KOU, Beijing (CN)

(73) Assignee: WISDRUG INNOVATIVE DRUG RESEARCH (BEIJING) CO.. LTD, Beijing (CN)

(21) Appl. No.: 18/827,915

(22) Filed: Sep. 9, 2024

Related U.S. Application Data
(63) Continuation of application No. PCT/CN2023/133223, filed on Nov. 22, 2023.

(30) Foreign Application Priority Data
Oct. 16, 2023 (CN) 202311332164.X

(51) Int. Cl. A61K 31/573; A61K 9/48; A61K 9/50

(52) U.S. Cl. CPC A61K 31/573; A61K 9/485; A61K 9/4858; A61K 9/4866; A61K 9/5047

(57) ABSTRACT

A prednisone enteric-coated preparation and a preparation method and use thereof are provided. The prednisone enteric-coated preparation includes: 5% to 35% of an active prednisone micro-enteric-coated preparation unit, such as an enteric-coated microsphere; and an inactive pharmaceutical adjuvant. The enteric-coated microsphere has an average particle size of 100 μm to 1,000 μm, and a dissolution behavior of the enteric-coated microsphere conforms to the provisions for enteric-coated preparations. The enteric-coated microsphere characteristic not only avoids the stimulation of prednisone to a gastric mucosa, but also makes the active ingredient quickly released and absorbed in an intestinal tract to prevent the active ingredient from being destroyed by intestinal floras. The prednisone enteric-coated preparation can be any dosage form such as a suspension or a capsule suitable for patients with difficult swallowing or normal patients.

### CROSS REFERENCE TO THE RELATED APPLICATIONS

This application is a continuation application of International Application No. PCT/CN2023/133223, filed on Nov. 22, 2022, which is based upon and claims priority to Chinese Patent Application No. 202311332164.X, filed on Oct. 16, 2023, the entire contents of which are incorporated herein by reference.

### TECHNICAL FIELD

The present application belongs to the field of pharmaceuticals, and specifically, the present application provides a prednisone enteric-coated microsphere preparation and a preparation method and use thereof.

### BACKGROUND

Due to its excellent anti-inflammatory and anti-allergy effects and few toxic and side effects, prednisone, the adrenocorticosteroid medicine, is widely used in clinical practice to treat diseases such as lupus erythematosus, rheumatoid arthritis, severe infections, and allergies. Prednisone is available merely in the dosage form of tablets on the market in China.

Prednisone is an intermediate-acting glucocorticoid drug most widely used in clinical practice. Due to pharmacological effects such as strong anti-inflammatory, anti-allergy, immunosuppressive, anti-rheumatism, and anti-tumor effects, prednisone is faced with the following problems in clinical practice: 1. Prednisone is frequently used for a long time in specific populations, such as children, the elderly, critically bedridden patients, and some patients using gastric tubes. However, the above-mentioned specific populations generally have impaired swallowing. The prednisone preparations on the market in China are merely ordinary tablets, and there is a lack of suitable dosage forms for the specific populations in clinical practice. Therefore, there is an urgent need for suitable dosage forms to improve medication compliance, safety, and scientificity for the above-mentioned population with impaired swallowing. 2. Prednisone itself has a bitter taste. Thus, when an ordinary liquid preparation such as an oral liquid developed from prednisone is taken, the bitter taste will continuously remain in the oral cavity due to the fat solubility of prednisone, which poses a huge challenge to the medication compliance of children. 3. The adverse reactions of prednisone are closely related to a course of treatment, a dosage, a type of medication, a usage, a route of administration, or the like, where gastric irritation is the most serious adverse reaction. Gastric irritation can cause peptic ulcers to progress rapidly, which manifest as sudden bleeding and perforation of the gastrointestinal tract, ulcerative esophagitis, vomiting, and other adverse reactions in severe cases.

However, side chains of cortisone steroids are easily degraded by some intestinal bacteria (see WEST HF. Prevention of peptic ulceration during corticosteroid therapy. Br Med J. 1959 Oct. 10; 2 (5153): 680 and Al-Sanea M M, Abdel-Hafez A A, Omar F A, Youssef A F. Biotransformation studies of prednisone using human intestinal bacteria Part II: Anaerobic incubation and docking studies. J Enzyme Inhib Med Chem. 2009 December; 24 (6): 1211-9), and thus a recurrence probability of arthritis treated with enteric-coated tablets is significantly higher than a recurrence probability of arthritis treated with gastric-soluble tablets. A change in the total excretion of a 17-hydroxycorticosteroid in urine can confirm that prednisolone cannot be totally absorbed. That is, if enteric-coated prednisolone preparations cannot be dissolved quickly after leaving the stomach, the enteric-coated prednisolone preparations will be inactivated by intestinal bacteria, which is a difficult point for the design of such drugs.

Based on the above reasons, the present disclosure develops prednisone into an enteric-coated microsphere preparation, which can allow medication convenience for children, avoid contact between the drug and a gastric mucosa, minimize the gastric irritation of the drug, and reduce the risk of gastric ulcer. The prednisone enteric-coated preparation of the present disclosure includes a prednisone enteric-coated microsphere as an active ingredient. An enteric-coated characteristic of the prednisone enteric-coated preparation can avoid stimulation to gastric mucosas, and a structural characteristic of the microsphere, “solid dispersion”, can make the active ingredient quickly released and absorbed in the human intestinal tract, which solves the problem that the prednisone enteric-coated preparations (tablets or capsules) prepared by the traditional enteric-coated preparation process release slowly and stay for a lone time in the intestinal tract, resulting in the destruction of active ingredients by intestinal flora and the failed achievement of an expected therapeutic effect. The preparation of the present disclosure can be prepared into a dosage form such as a dry suspension, a granule, or a dispersible tablet suitable for patients with difficult swallowing, such as children, the elderly, critically bedridden patients, and patients without self-help skills, and can also be prepared into a capsule or a tablet suitable for patients who can take medicine normally.

### SUMMARY

Enteric-coated dry suspensions and enteric-coated dispersible tablets have both the characteristics such as high portability, convenient transportation, and excellent stability of solid preparations and the advantages of liquid preparations, which increase the compliance for patients with difficult swallowing (such as children and the elderly). When a drug is embedded in microspheres, a bitter taste can be completely masked to improve the medication compliance for children. A liquid preparation can be quickly absorbed in an intestinal tract through a pylorus, which fits a circadian rhythm of glucocorticoid secretion, improves the safety and effectiveness of medication, and has obvious clinical advantages. According to an equivalency trial, the liquid preparation has an equivalent effect to the gastric-soluble tablets currently on the market.

In order to solve the above problems, in a first aspect, the present application provides a prednisone enteric-coated preparation, including a prednisone micro-enteric-coated preparation unit as an active ingredient and a pharmaceutical adjuvant as an inactive ingredient.

Further, the prednisone micro-enteric-coated preparation unit is a prednisone enteric-coated microsphere, and the prednisone enteric-coated microsphere includes 10% to 40% of prednisone and 60% to 90% of hypromellose phthalate in mass percentages.

Further, the prednisone enteric-coated microsphere includes 25% to 35% of the prednisone and 65% to 75% of the pharmaceutical enteric polymer material in mass percentages.

Further, the prednisone enteric-coated microsphere includes 30% of the prednisone and 70% of the hypromellose phthalate in mass percentages.

Further, the prednisone enteric-coated microsphere has an average particle size of 100 μm to 1,000 μm, an encapsulation rate of greater than 80%, a 2 h dissolution rate of less than 10% in an acid, and a 30 min dissolution rate of greater than 80% in a pH 6.8 buffer.

Further, the enteric-coated preparation is an enteric-coated suspension, an enteric-coated granule, an enteric-coated capsule, or an enteric-coated dispersible tablet.

Further, the enteric-coated dry suspension includes the prednisone enteric-coated microsphere as an active ingredient and a suspending agent, corrigent, filler, and fragrance as a pharmaceutically-acceptable adjuvant.

Further, the suspending agent is selected from one or more of xanthan gum, sodium carboxymethyl cellulose, colloidal microcrystalline cellulose, hypromellose, gum arabic, and tragacanth, and a weight percentage of the suspending agent is 1.0% to 10.0%.

Further, the corrigent is selected from one or more of sucrose, aspartame, acesulfame potassium, sucralose, sodium saccharin, citric acid, and tartaric acid, and a weight percentage of the corrigent is 1.0% to 10.0%.

Further, the filler is selected from one or more of sucrose, corn starch, microcrystalline cellulose, lactose, and maltitol, and a weight percentage of the filler is 10.0% to 90.0%.

Further, the fragrance is selected from one or more of a peppermint fragrance, a peach fragrance, a strawberry fragrance, an orange fragrance, and a lemon fragrance, and a weight percentage of the fragrance is 0.1% to 5.0%.

Further, the pharmaceutically-acceptable adjuvant includes the following components in weight percentage contents:

| | |
|---|---|
| Xanthan gum | 4.0-7.0% |
| Lactose | 10.0-90.0% |
| Aspartame | 1.0-2.0% |
| Acesulfame potassium | 1.0-2.0% |
| Citric acid | 2.0-5.0% |
| Peppermint fragrance | 0.1-0.5% |
| Peach fragrance | 0.5-1.0% |

Further, the enteric-coated preparation includes the following components in weight percentages:

| | |
|---|---|
| Prednisone enteric-coated microsphere | 15.00% |
| Xanthan gum | 6.00% |
| Lactose | 72.40% |
| Aspartame | 1.50% |
| Acesulfame potassium | 1.00% |
| Citric acid | 3.00% |
| Peppermint fragrance | 0.30% |
| Peach fragrance | 0.80% |

Further, the enteric-coated capsule includes the prednisone enteric-coated microsphere as an active ingredient and a filler as a pharmaceutically-acceptable adjuvant.

Further, the filler is selected from one or more of sucrose, corn starch, microcrystalline cellulose, lactose, and maltitol, and a weight percentage of the filler is 10.0% to 90.0%.

Further, a content in the enteric-coated capsule includes the following components in weight percentages:

| | |
|---|---|
| Prednisone enteric-coated microsphere | 22% |
| Granulated lactose | 78% |

A capsule shell is gastric-soluble.

Further, the enteric-coated dispersible tablet includes the prednisone enteric-coated microsphere as an active ingredient and a filler, disintegrant, binder, and lubricant as a pharmaceutically-acceptable adjuvant.

Further, the disintegrant is one or more of pharmaceutically-acceptable crospovidone, sodium carboxymethyl starch, croscarmellose sodium, microcrystalline cellulose, dry starch, and low-substituted hydroxypropyl cellulose, and a weight percentage of the disintegrant is 1.0% to 7.0%.

Further, the binder is one or more of pharmaceutically-acceptable povidone, hypromellose, hydroxypropyl cellulose, sodium carboxymethyl cellulose, and a starch slurry, and a weight percentage of the binder is 1.0% to 7.0%.

Further, the lubricant is one or more of pharmaceutically-acceptable magnesium stearate, talc, colloidal silicon dioxide, and stearic acid, and a weight percentage of the lubricant is 0.1% to 1.0%.

Further, the pharmaceutically-acceptable adjuvant includes the following components in weight percentage contents:

| | |
|---|---|
| Lactose | 10.0-40.0% |
| Microcrystalline cellulose | 10.0-50.0% |
| Povidone K30 | 2.0-5.0% |
| Croscarmellose sodium | 2.0-5.0% |
| Crospovidone | 2.0-5.0% |
| Colloidal silicon dioxide | 0.01-0.1% |

Further, the enteric-coated preparation includes the following components in weight percentages:

| | |
|---|---|
| Prednisone enteric-coated microsphere | 10% |
| Lactose | 35% |
| Microcrystalline cellulose | 46.95% |
| Povidone K30 | 2% |
| Croscarmellose sodium | 3% |
| Crospovidone | 3% |
| Colloidal silicon dioxide | 0.05% |

In a second aspect, the present application provides a preparation method of the enteric-coated dry suspension described above, including: preparing the prednisone enteric-coated microsphere; mixing ½ of the xanthan gum, the lactose, the aspartame, the acesulfame potassium, and the citric acid in a wet granulator, adding an appropriate amount of purified water to prepare a soft material, subjecting the soft material to fluidized bed drying and sieving; adding the remaining ½ of the xanthan gum, the peppermint fragrance, and the peach fragrance to mix, and finally mixing with the prednisone enteric-coated microsphere thoroughly.

In a third aspect, the present application provides a preparation method of the enteric-coated capsule described above, including: preparing the prednisone enteric-coated microsphere; thoroughly mixing the granulated lactose with the prednisone enteric-coated microsphere to obtain a mixture; and filling the mixture in a 5# gastric-soluble capsule shell to obtain the prednisone enteric-coated capsule.

In a fourth aspect, the present application provides a preparation method of the enteric-coated dispersible tablet described above, including: preparing the prednisone enteric-coated microsphere; mixing the lactose, the microcrystalline cellulose, and the povidone K30 in a wet granulator, adding an appropriate amount of purified water to prepare a soft material, subjecting the soft material to fluidized bed drying and sieving; adding the croscarmellose sodium, the crospovidone, and the colloidal silicon dioxide to mix thoroughly, and finally mixing with the prednisone enteric-coated microsphere thoroughly, and obtaining the prednisone enteric-coated dispersible tablet by tableting with a rotary tablet press.

A preparation method of the prednisone micro-enteric-coated preparation unit includes: dissolving the pharmaceutical enteric polymer material in an organic solvent, adding a prednisone raw material to produce an oil phase, adding the oil phase to an aqueous phase containing an emulsifier, stirring to produce an emulsion droplet, and solidifying the emulsion droplet to obtain the microsphere. The organic solvent is selected from one or more of methyl acetate, ethyl acetate, dichloromethane, ethanol, and n-heptane; and/or the emulsifier is selected from one or more of Tween, Span, sodium dodecyl sulfate, and polyvinyl alcohol.

The enteric-coated preparation of the present application is used for allergic and autoimmune inflammatory diseases. The enteric-coated preparation is suitable for an allergic disease, an autoimmune inflammatory disease, acute leukemia, or malignant lymphoma; and the allergic disease is a connective tissue disease, systemic lupus erythematosus, severe polymyositis, severe bronchial asthma, dermatomyositis, or vasculitis.

In a fifth aspect, the present application provides a use of the enteric-coated preparation described above in preparation of a pharmaceutical preparation for an allergic disease, an autoimmune inflammatory disease, acute leukemia, or malignant lymphoma, where the allergic disease is a connective tissue disease, systemic lupus erythematosus, severe polymyositis, severe bronchial asthma, dermatomyositis, or vasculitis.

In a sixth aspect, the present application provides a method for treating an allergic disease, an autoimmune inflammatory disease, acute leukemia, or malignant lymphoma, including: administering the enteric-coated preparation described above to a patient, where the allergic disease is a connective tissue disease, systemic lupus erythematosus, severe polymyositis, severe bronchial asthma, dermatomyositis, or vasculitis; and an administration dosage is 5 mg/d to 100 mg/d and preferably 10 mg/d to 60 mg/d, and an administration frequency is 1 time/d to 12 times/d and preferably 1 time/d to 6 times/d.

The enteric-coated preparation of the present application is particularly suitable for patients with difficult swallowing, and the difficult swallowing may be caused by oropharyngeal, esophageal, and psychological factors or the like. Clinically-known difficult swallowing includes, but is not limited to, difficult swallowing in children, difficult swallowing in the elderly, difficult swallowing caused by psychological factors, difficult swallowing during recovery after surgery, difficult swallowing caused by tumors or hyperplasia, difficult swallowing caused by birth defects, difficult swallowing caused by esophageal diseases (physical clogging or dysperistalsis), general muscular diseases, and brain or nervous system diseases, or the like.

The prednisone enteric-coated preparation, especially the enteric-coated dry suspension, the enteric-coated granule, and the enteric-coated dispersible tablet in the present application, are very suitable for patients with difficult swallowing due to excellent taste characteristics and swallowing performance. The prednisone enteric-coated preparation can be used for treating allergic and autoimmune inflammatory diseases. The prednisone enteric-coated preparation is suitable for allergic diseases such as connective tissue diseases, systemic lupus erythematosus, severe polymyositis, severe bronchial asthma, dermatomyositis, and vasculitis, acute leukemia, malignant lymphoma, or the like.

### BRIEF DESCRIPTION OF THE DRAWINGS

FIG. 1 is a scanning electron microscopy image of a prednisone enteric-coated microsphere in Example 1;

FIG. 2 is a microscopy image of the prednisone enteric-coated microsphere prepared with hypromellose phthalate as a matrix material in Example 1;

FIG. 3 is a microscopy image of a prednisone enteric-coated microsphere prepared with a Eudragit L100 matrix material in Example 2;

FIG. 4 shows a particle size distribution of the prednisone enteric-coated microsphere in Example 1;

FIG. 5 shows concentration-time curves of prednisone in subject 101 during a fasting test;

FIG. 6 shows concentration-time curves of prednisone in subject 102 during a fasting test;

FIG. 7 shows concentration-time curves of prednisone in subject 103 during a fasting test;

FIG. 8 shows concentration-time curves of prednisone in subject 104 during a fasting test;

FIG. 9 shows concentration-time curves of prednisone in subject 105 during a fasting test;

FIG. 10 shows concentration-time curves of prednisone in subject 106 during a fasting test;

FIG. 11 shows concentration-time curves of prednisone in subject 107 during a fasting test;

FIG. 12 shows concentration-time curves of prednisone in subject 108 during a fasting test;

FIG. 13 shows concentration-time curves of prednisone in subject 109 during a fasting test;

FIG. 14 shows concentration-time curves of prednisone in subject 110 during a fasting test;

FIG. 15 shows mean concentration-time curves of prednisone in subjects during a fasting test;

FIG. 16 shows concentration-time curves of prednisolone in the subject 101 during a fasting test;

FIG. 17 shows concentration-time curves of prednisolone in the subject 102 during a fasting test;

FIG. 18 shows concentration-time curves of prednisolone in the subject 103 during a fasting test;

FIG. 19 shows concentration-time curves of prednisolone in the subject 104 during a fasting test;

FIG. 20 shows concentration-time curves of prednisolone in the subject 105 during a fasting test;

FIG. 21 shows concentration-time curves of prednisolone in the subject 106 during a fasting test;

FIG. 22 shows concentration-time curves of prednisolone in the subject 107 during a fasting test;

FIG. 23 shows concentration-time curves of prednisolone in the subject 108 during a fasting test;

FIG. 24 shows concentration-time curves of prednisolone in the subject 109 during a fasting test;

FIG. 25 shows concentration-time curves of prednisolone in the subject 110 during a fasting test; and

FIG. 26 shows mean concentration-time curves of prednisolone in subjects during a fasting test.

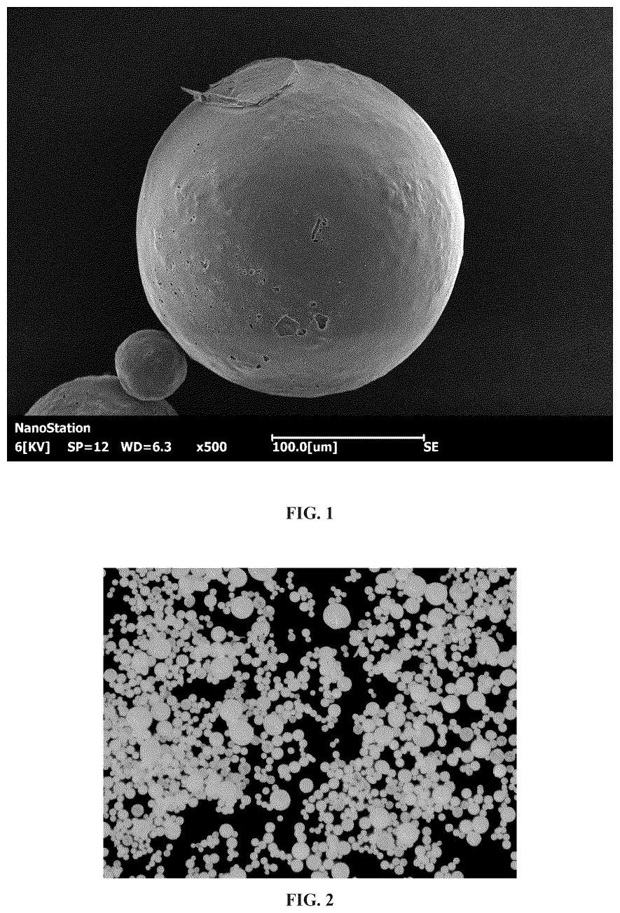

### DETAILED DESCRIPTION OF THE EMBODIMENTS

The present disclosure is described in detail below in conjunction with specific examples. The following examples are provided merely for illustration, and the protection scope of the present disclosure is defined by the claims and is not limited to the following examples. Unless otherwise specified, the percentages or proportions in the formulas of the following examples are calculated based on weights.

### Example 1

According to the present disclosure, a prednisone enteric-coated microsphere was prepared according to the following formulas:

A prednisone enteric-coated microsphere as an active ingredient included:

| | |
|---|---|
| Prednisone | 30% |
| Hypromellose phthalate | 70% |

A preparation method of the prednisone enteric-coated microsphere was an emulsification-solvent evaporation method as follows: An enteric-coating material of hypromellose phthalate was dissolved in ethyl acetate and ethanol, a prednisone raw material was added to produce an oil phase, then the oil phase was added to an aqueous phase including polyvinyl alcohol, stirring was conducted to produce an emulsion droplet, and the emulsion droplet was solidified to produce the microsphere. In other formulas, the preparation method was basically the same as above except for components and amounts thereof.

A prednisone enteric-coated dry suspension included:

| | |
|---|---|
| Prednisone enteric-coated microsphere | 15.00% |
| Xanthan gum | 6.00% |
| Lactose | 72.40% |
| Aspartame | 1.50% |
| Acesulfame potassium | 1.00% |
| Citric acid | 3.00% |
| Peppermint fragrance | 0.30% |
| Peach fragrance | 0.80% |

A preparation method was as follows: ½ of xanthan gum, lactose, aspartame, acesulfame potassium, and citric acid were taken and mixed in a wet granulator, an appropriate amount of purified water was added to prepare a wet particle, and the wet particle was subjected to fluidized bed drying with an inlet air temperature of 50° C. to 60° C. until a moisture content was less than 3%, then sieved, and transferred to a mixing machine. The remaining ½ of the xanthan gum, a peppermint fragrance, and a peach fragrance were added to the mixing machine, mixing was conducted, the prednisone enteric-coated microsphere was added, and thorough mixing was conducted to obtain the prednisone enteric-coated dry suspension. The prednisone enteric-coated dry suspension was filled in a two-chamber oral liquid bottle for easy administration by a patient.

A prednisone enteric-coated capsule included:

| | |
|---|---|
| Prednisone enteric-coated microsphere | 22% |
| Granulated lactose | 78% |

A preparation method was as follows: Granulated lactose with D90 of about 300 μm and the prednisone enteric-coated microsphere were thoroughly mixed and then filled in a 5# gastric-soluble capsule.

A prednisone enteric-coated microsphere dispersible tablet included the following components at contents as follows:

| | |
|---|---|
| Prednisone enteric-coated microsphere | 10% |
| Lactose | 35% |
| Microcrystalline cellulose | 46.95% |
| Povidone K30 | 2% |
| Croscarmellose sodium | 3% |
| Crospovidone | 3% |
| Colloidal silicon dioxide | 0.05% |

In the table, a content of a component refers a percentage of an amount of the component in a total amount of the components.

A preparation method was as follows: A prednisone enteric-coated microsphere was prepared. Lactose, microcrystalline cellulose, and povidone K30 were mixed in a wet granulator, an appropriate amount of purified water was added to prepare a soft material, the soft material was subjected to fluidized bed drying and sieving, croscarmellose sodium, crospovidone, and colloidal silicon dioxide were added and mix thoroughly, and finally mixed with the prednisone enteric-coated microsphere thoroughly and tableted by a rotary tablet press to obtain the prednisone enteric-coated dispersible tablet.

### Example 2

A prednisone enteric-coated microsphere as an active ingredient included:

| | |
|---|---|
| Prednisone | 30% |
| Eudragit L100 | 70% |

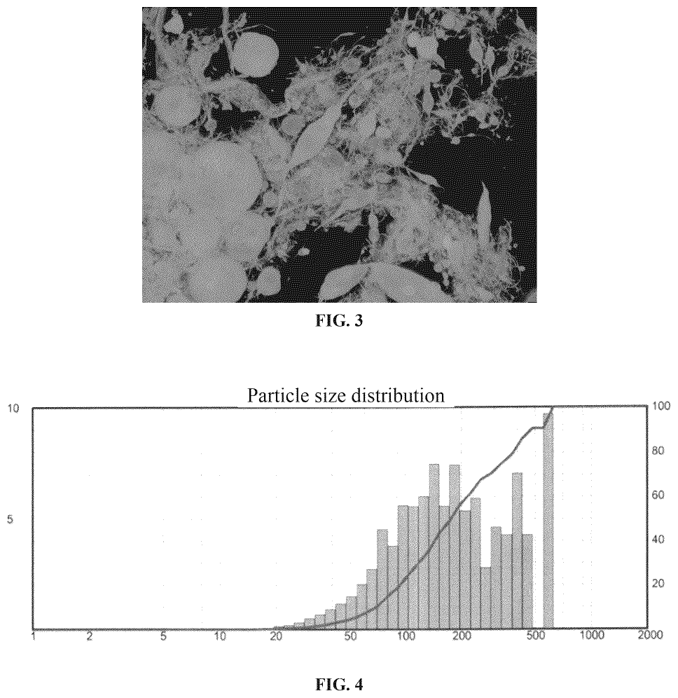

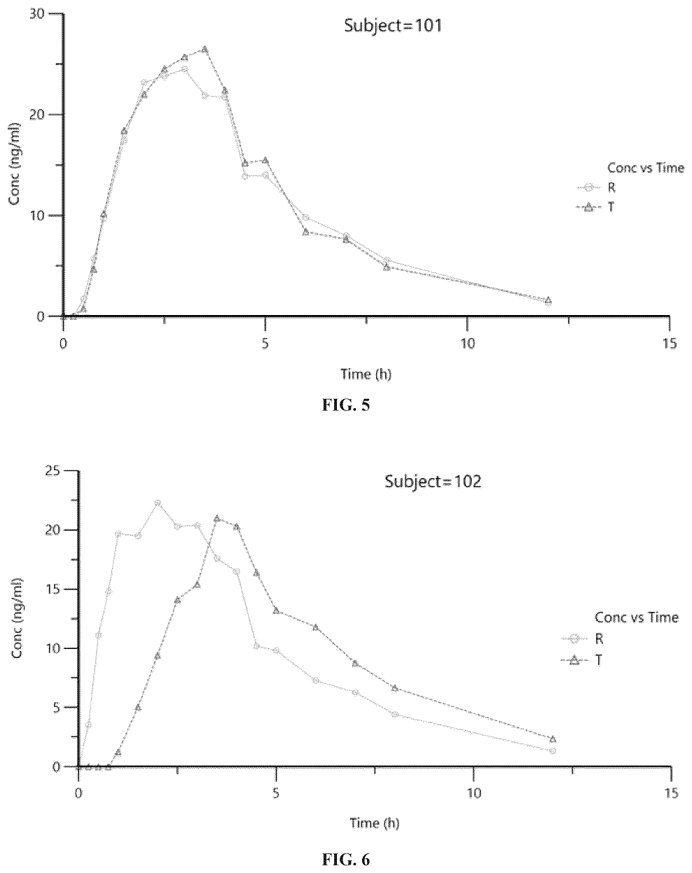

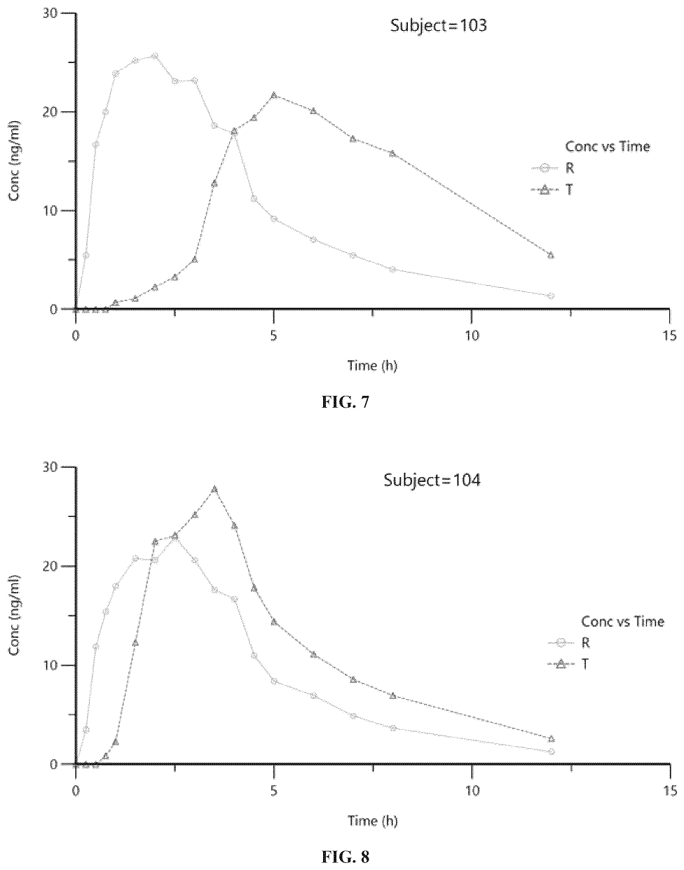

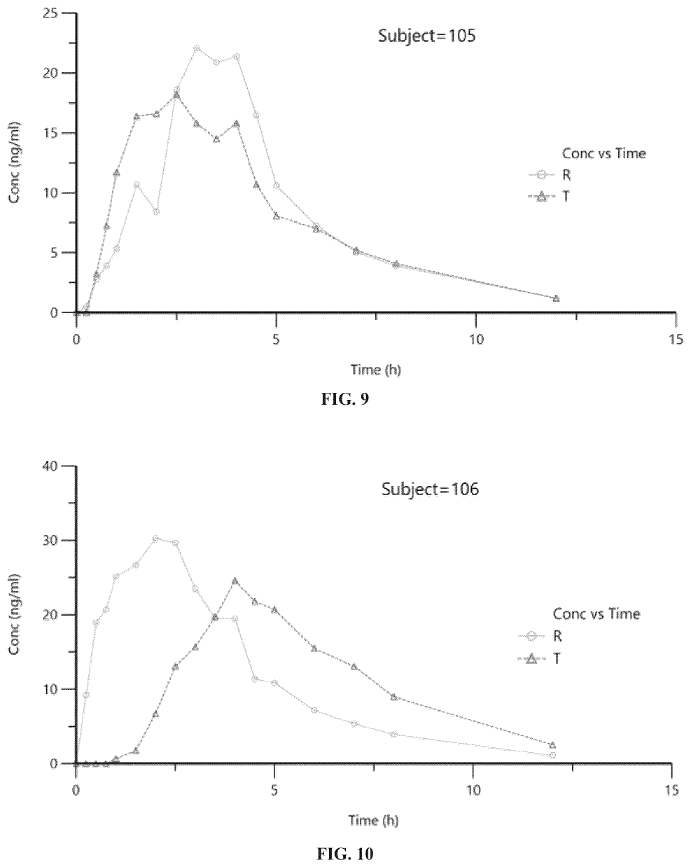

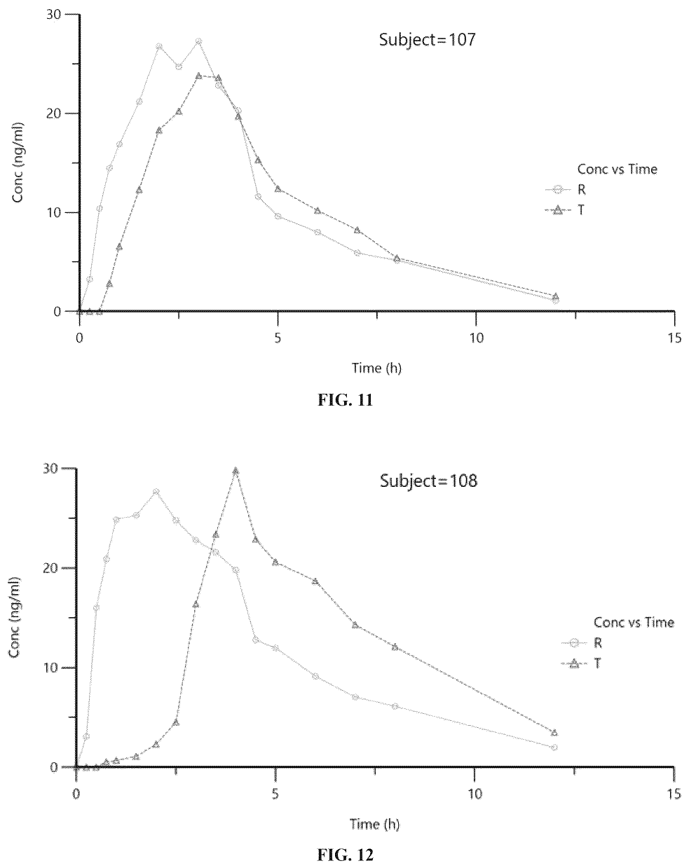

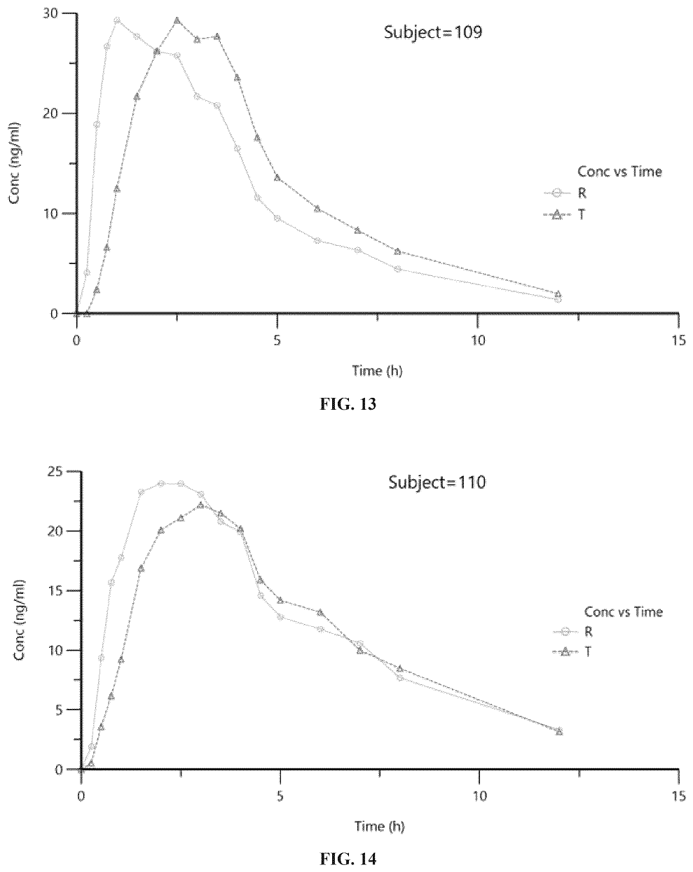

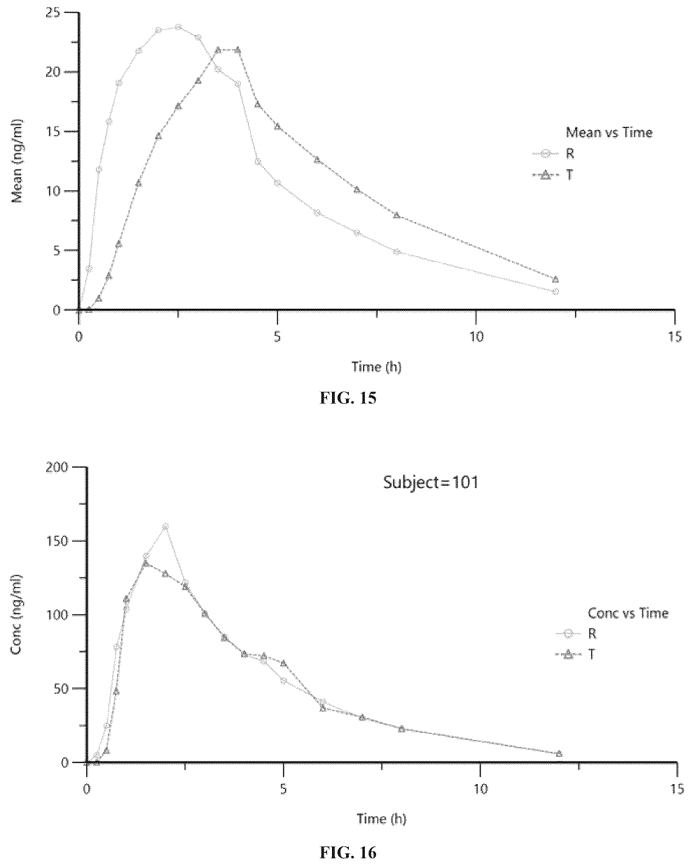

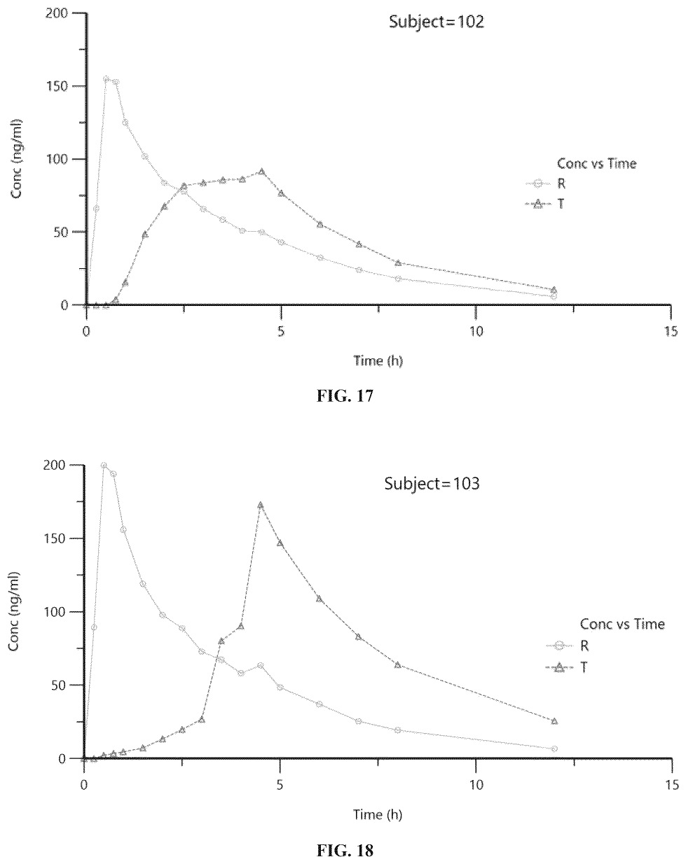

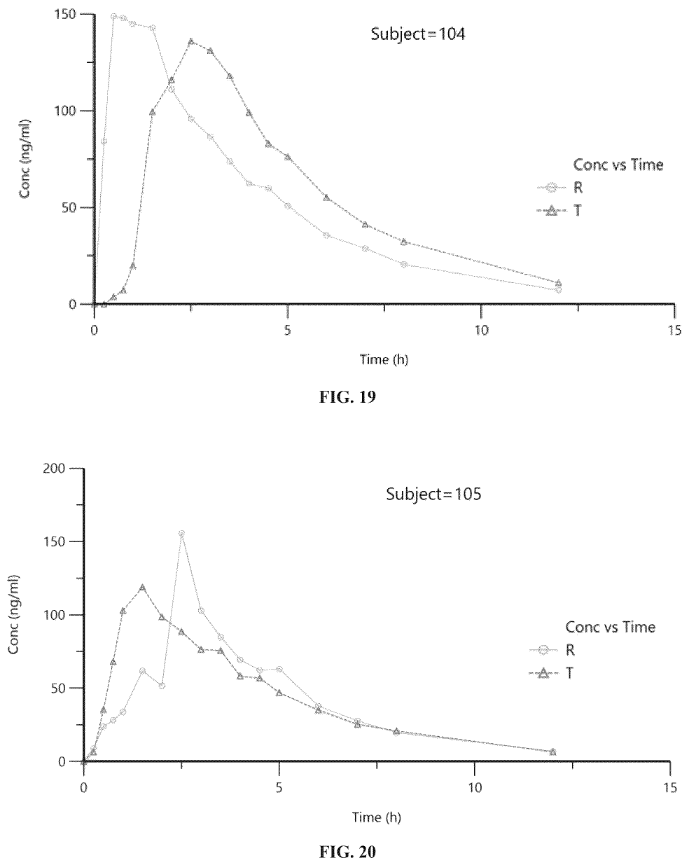

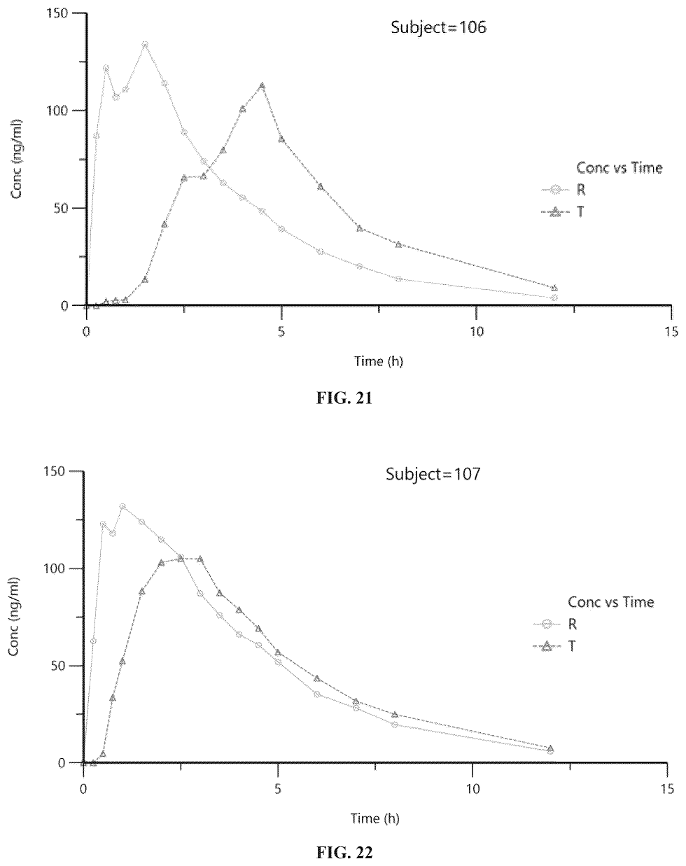

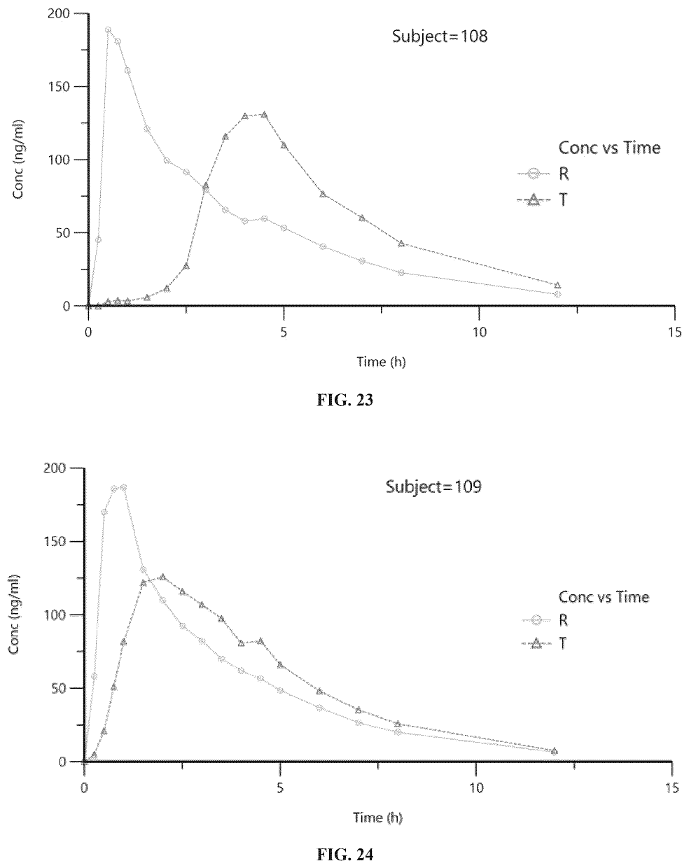

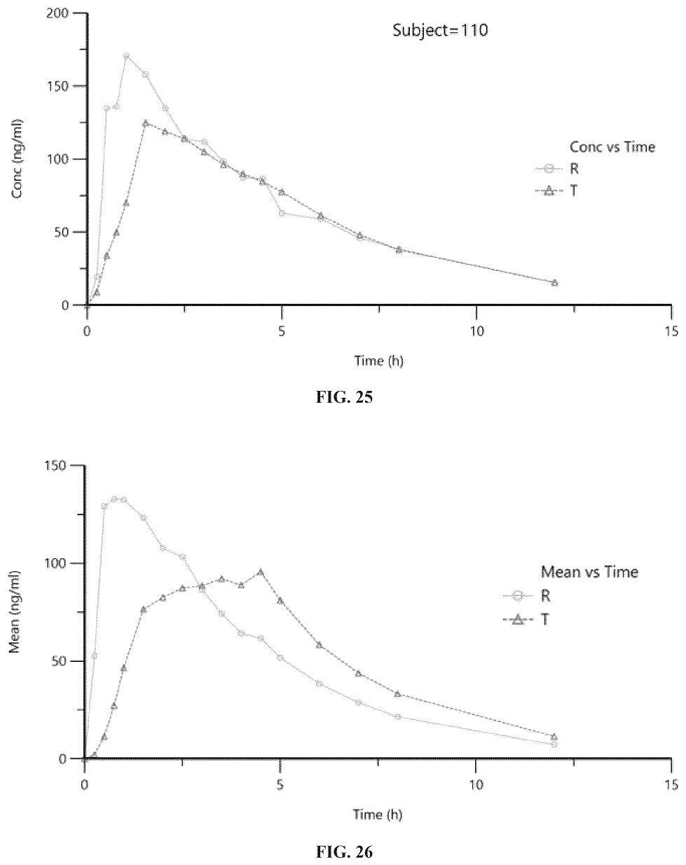

A prednisone enteric-coated microsphere produced by the formula of prednisone+Eudragit L100 included a large number of flocci. A microscopy image was shown in FIG. 3. It can be seen that the formula adopting Eudragit L100 is infeasible.

### Example 3

A prednisolone enteric-coated microsphere was prepared according to the following formula:

A prednisolone enteric-coated microsphere as an active ingredient included:

| | |
|---|---|
| Prednisolone | 30% |
| Hypromellose phthalate | 70% |

A prednisolone enteric-coated dry suspension included the following ingredients:

| | |
|---|---|
| Prednisolone enteric-coated | 15% |
| microsphere | |
| Xanthan gum | 6.00% |
| Lactose | 72.40% |
| Aspartame | 1.50% |
| Acesulfame potassium | 1.00% |
| Citric acid | 3.00% |
| Peppermint fragrance | 0.30% |
| Peach fragrance | 0.80% |

A preparation method of the prednisolone enteric-coated dry suspension was as follows: ½ of xanthan gum, lactose, aspartame, acesulfame potassium, and citric acid were taken and mixed in a wet granulator, an appropriate amount of purified water was added to prepare a wet particle, and the wet particle was subjected to fluidized bed drying with an inlet air temperature of 50° C. to 60° C. until a moisture content was less than 3%, then sieved, and transferred to a mixing machine. The remaining ½ of the xanthan gum, a peppermint fragrance, and a peach fragrance were added to the mixing machine, mixing was conducted, the prednisolone enteric-coated microsphere was added, and thorough mixing was conducted.

### Example 4

A prednisone enteric-coated microsphere as an active ingredient included:

| | |
|---|---|
| Prednisone | 50% |
| Hypromellose phthalate | 50% |

A prednisone enteric-coated dry suspension included the following ingredients:

| | |
|---|---|
| Prednisone enteric-coated microsphere | 10.00% |
| Xanthan gum | 6.00% |
| Lactose | 77.40% |
| Aspartame | 1.50% |
| Acesulfame potassium | 1.00% |
| Citric acid | 3.00% |
| Peppermint fragrance | 0.30% |
| Peach fragrance | 0.80% |

A preparation method of the prednisone enteric-coated dry suspension was as follows: ½ of xanthan gum, lactose, aspartame, acesulfame potassium, and citric acid were taken and mixed in a wet granulator, an appropriate amount of purified water was added to prepare a wet particle, and the wet particle was subjected to fluidized bed drying with an inlet air temperature of 50° C. to 60° C. until a moisture content was less than 3%, then sieved, and transferred to a mixing machine. The remaining ½ of the xanthan gum, a peppermint fragrance, and a peach fragrance were added to the mixing machine, mixing was conducted, the prednisone enteric-coated microsphere was added, and thorough mixing was conducted to obtain the prednisone enteric-coated dry suspension. The prednisone enteric-coated dry suspension was filled in a two-chamber oral liquid bottle for easy administration by a patient.

### Example 5

In this example, the lactose in the prednisone enteric-coated microsphere dry suspension in Example 1 was replaced with mannitol. The prednisone enteric-coated dry suspension included:

| | |
|---|---|
| Prednisone enteric-coated microsphere | 15% |
| Xanthan gum | 6.00% |
| Mannitol | 72.40% |
| Aspartame | 1.50% |
| Acesulfame potassium | 1.00% |
| Citric acid | 3.00% |
| Peppermint fragrance | 0.30% |
| Peach fragrance | 0.80% |

A preparation method of the prednisone enteric-coated dry suspension was as follows: ½ of xanthan gum, mannitol, aspartame, acesulfame potassium, and citric acid were taken and mixed in a wet granulator, an appropriate amount of purified water was added to prepare a wet particle, and the wet particle was subjected to fluidized bed drying with an inlet air temperature of 50° C. to 60° C. until a moisture content was less than 3%, then sieved, and transferred to a mixing machine. The remaining ½ of the xanthan gum, a peppermint fragrance, and a peach fragrance were added to the mixing machine, mixing was conducted, the prednisone enteric-coated microsphere was added, and thorough mixing was conducted to obtain the prednisone enteric-coated dry suspension. The prednisone enteric-coated dry suspension was filled in a two-chamber oral liquid bottle to obtain the prednisone enteric-coated microsphere dry suspension.

### Example 6

In this example, the lactose in the prednisone enteric-coated microsphere dry suspension in Example 1 was replaced with sorbitol. The prednisone enteric-coated dry suspension included:

| | |
|---|---|
| Prednisone enteric-coated microsphere | 15% |
| Xanthan gum | 6.00% |
| Sorbitol | 72.40% |
| Aspartame | 1.50% |
| Acesulfame potassium | 1.00% |
| Citric acid | 3.00% |
| Peppermint fragrance | 0.30% |
| Peach fragrance | 0.80% |

A preparation method of the prednisone enteric-coated dry suspension was as follows: ½ of xanthan gum, sorbitol, aspartame, acesulfame potassium, and citric acid were taken and mixed in a wet granulator, an appropriate amount of purified water was added to prepare a wet particle, and the wet particle was subjected to fluidized bed drying with an inlet air temperature of 50° C. to 60° C. until a moisture content was less than 3%, then sieved, and transferred to a mixing machine. The remaining ½ of the xanthan gum, a peppermint fragrance, and a peach fragrance were added to the mixing machine, mixing was conducted, the prednisone enteric-coated microsphere was added, and thorough mixing was conducted to obtain the prednisone enteric-coated dry suspension. The prednisone enteric-coated dry suspension was filled in a two-chamber oral liquid bottle to obtain the prednisone enteric-coated microsphere dry suspension.

### Example 7 Performance Test Results of Each Preparation

### In Vitro Dissolution Curves:

The enteric-coated preparation dissolution determination method in the Chinese Pharmacopoeia of 2020 edition was adopted. An intestinal alkaline environment was simulated by first adopting a pH 1.0 hydrochloric acid medium for 2 h and then replacing the dissolution medium with a pH 6.8 phosphate buffered saline to detect a dissolution release of each product.

TABLE 1
In vitro dissolution data of samples in the examples

| | pH 1.0 | | | | | | |
|---|---|---|---|---|---|---|---|
| | hydrochloric | | | | | | |
| | acid medium | pH 6.8 phosphate buffered saline medium | | | | | |
| In vitro dissolution | Dissolution time | | | | | | |
| results (%) | 2 h | 5 min | 10 min | 15 min | 30 min | 45 min | 60 min |
| Prednisone enteric- | 2.1 | 53.7 | 79.8 | 87.8 | 95.7 | 98.0 | 99.2 |
| coated microsphere dry | | | | | | | |
| suspension of Example 1 | | | | | | | |
| Prednisone enteric- | 2.3 | 43.3 | 72.7 | 87.7 | 95.5 | 96.2 | 96.0 |
| coated microsphere | | | | | | | |
| capsule of Example 1 | | | | | | | |
| Prednisone enteric- | 2.1 | 45.1 | 73.9 | 88.8 | 97.6 | 98.3 | 97.8 |
| coated microsphere | | | | | | | |
| dispersible tablet of | | | | | | | |
| Example 1 | | | | | | | |
| Example 3 | 10.5 | 45.3 | 79.5 | 89.9 | 97.2 | 98.6 | 99.1 |
| Example 4 | 12.4 | 52.5 | 80.8 | 90.6 | 98.4 | 99.2 | 99.6 |

The results show that the in vitro dissolution of each of the prednisone enteric-coated microsphere dry suspension, the prednisone enteric-coated microsphere capsule, and the prednisone enteric-coated microsphere dispersible tablet meets the dissolution requirements of enteric-coated preparations, and a dissolution rate in the acid is less than 10%. However, an in vitro dissolution rate of the prednisolone enteric-coated microsphere prepared with the same formula in the acid is greater than 10%, which does not meet the dissolution requirements of enteric-coated preparations. When a ratio of prednisone to hypromellose phthalate increases to 1:1 in Example 4, the corresponding in vitro dissolution cannot meet the dissolution requirements of enteric-coated preparations.

### Pharmacokinetics in Dogs

The pharmacokinetic characteristics and equivalency of the prednisone enteric-coated capsule, the prednisone enteric-coated dry suspension, and an FDA-commercialized prednisone tablet (5 mg, trade name: Prednisone®) in beagle dogs were investigated by a three-cycle and self-cross-controlled experimental design. Male beagle dogs were orally administered with the prednisone enteric-coated dry suspension (microsphere) (T1), the prednisone enteric-coated capsule (T2), and the commercialized prednisone tablet as a reference preparation (R, Batch No.: PX113A) once at 5 mg/dog, and then a concentration of prednisone in plasma was measured by liquid chromatography with tandem mass spectrometry (LC-MS/MS) in three cycles.

TABLE 2
Comparison of parameters for pharmacokinetics in dogs

| Key parameters for | Cₘₐₓ | AUC₀₋ₜ | AUC_{0-∞} |
|---|---|---|---|
| pharmacokinetics in dogs | (ng/mL) | (h*ng/mL) | (h*ng/mL) |
| Commercially-available | 21.1 | 65.9 | 69.8 |
| prednisone tablet | | | |
| Dry suspension of | 18.9 | 67.6 | 70.6 |
| Example 1 | | | |
| Capsule of Example 1 | 18.6 | 70.9 | 74.9 |
| Dispersible tablet of | 21.5 | 71.3 | 75.0 |
| Example 1 | | | |
| Example 5 | 18.4 | 44.9 | 47.8 |
| Example 6 | 16.3 | 50.4 | 61.2 |

TABLE 3
T-test for pharmacokinetic parameters of each example
and the commercially-available prednisone tablet

| | P value | | | | |
|---|---|---|---|---|---|
| | Dry | | Dispersible | | |
| Pharmacokinetic | suspension of | Capsule of | tablet of | Example | Example |
| parameters | Example 1 | Example 1 | Example 1 | 5 | 6 |
| Cₘₐₓ (ng/mL) | 0.429 | 0.324 | 0.638 | 0.320 | 0.013 |
| AUC₀₋ₜ(h*ng/mL) | 0.851 | 0.514 | 0.506 | 0.005 | 0.006 |
| AUC_{0-∞} (h*ng/mL) | 0.921 | 0.506 | 0.497 | 0.008 | 0.005 |

TABLE 4
Bioavailability of each example and the
commercially-available prednisone tablet

| Bioavailability (%) | AUC₀₋ₜ | AUC_{0-∞} |
|---|---|---|
| Dry suspension of Example 1 | 102.6 | 101.1 |
| Capsule of Example 1 | 107.6 | 107.3 |
| Dispersible tablet of Example 1 | 108.2 | 107.4 |
| Example 5 | 68.1 | 68.5 |
| Example 6 | 76.5 | 87.7 |

According to results: There is no significant difference between the two preparations in Example 1 and the reference preparation in terms of Cₘₐₓ and AUC (p<0.05 indicates a significant difference), indicating that the two test preparations have very similar pharmacokinetic behaviors to the commercially-available preparation in male beagle dogs. The two test preparations have a higher bioavailability than the commercially-available preparation during in vivo absorption, indicating that the enteric-coated microsphere of the present disclosure has better in vivo absorption than the commercially-available preparation. The pharmacokinetic parameter AUCs of the samples in Example 5 (mannitol is adopted in the formula) and Example 6 (sorbitol is adopted in the formula) are significantly smaller than the pharmacokinetic parameter AUC of the commercially-available preparation, indicating that mannitol and sorbitol have a significant impact on the in vivo absorption of prednisone and the formula of the present disclosure does not affect the in vivo absorption of prednisone.

### Taste Evaluation—Dissolution in Simulated Saliva

A quantitative taste evaluation/in vitro dissolution experiment based on analytical methods was conducted, which was mainly as follows: pharmaceutical components in an aqueous medium were detected in a short period of time (for example, no more than 3 mL of simulated saliva), the effectiveness of coating and complexation in a preparation were measured, and whether the taste-masking technology is up to standard was indirectly evaluated.

An experiment method for in vitro dissolution in the simulated saliva was as follows: A commercially-available prednisone tablet, the prednisone enteric-coated microsphere dry suspension, and the prednisone enteric-coated dispersible tablet each were prepared into a suspension. 2 mL of the suspension was taken, 3 mL of the artificial saliva was added to the suspension to obtain a mixed solution, and timing was started. A content of prednisone in the mixed solution was detected at 30 s, 1 min, 2 min, 3 min, and 5 min.

TABLE 5
Concentrations of the prednisone raw material and the preparations
in the dissolution solution (artificial saliva)

| | | Dissolution amount (μg/mL) | | |
|---|---|---|---|---|
| | | | Prednisone | Prednisone |
| | | Commercially- | enteric-coated | enteric-coated |
| | | available | microsphere dry | dispersible |
| | | prednisone | suspension | tablet |
| Time | | tablet | of Example | of Example 1 |
| 30 | s | 136.57 | 0.77 | 0.73 |
| 1 | min | 131.93 | 1.03 | 1.01 |
| 2 | min | 127.66 | 0.98 | 1.00 |
| 3 | min | 126.35 | 1.32 | 1.24 |
| 5 | min | 133.71 | 1.24 | 1.22 |

### Taste Evaluation—Electronic Tongue

With a taste analysis system of Japan INSENT, a taste was evaluated by detecting a change in a membrane potential caused by an electrostatic interaction or a hydrophobic interaction between each flavor substance and an artificial lipid membrane.

TABLE 6
Test results of a bitterness sensor and a conventional bitterness and astringency sensor

| | B- | Aftertaste- | Aftertaste- | H- | | |
|---|---|---|---|---|---|---|
| CH | bitterness 2 | B | A | bitterness | Bitterness | Astringency |
| RE* | 0 | 0 | 0 | 0 | 0 | 0 |
| Microsphere of Example 1 | −0.27 | −0.33 | −0.03 | −0.08 | −0.23 | −15.24 |
| Commercially-available | −1.45 | −0.26 | 0.01 | −0.26 | 4.82 | −11.54 |
| prednisone tablet | | | | | | |
| Example 3 | −0.07 | −0.3 | −0.01 | −0.07 | 1.17 | −15.34 |
| Example 4 | −0.18 | −0.29 | 0.01 | −0.06 | 1.04 | −15.50 |
| Example 5 | −0.75 | −0.26 | 0.02 | −0.11 | 0.07 | −15.47 |
| Example 6 | −0.4 | −0.15 | 0.03 | −0.07 | 0.98 | −15.34 |

Notes: *: With a reference solution as a tastelessness point, a value lower than 0 indicates that a sensor response value is low, and no analysis is conducted.

Conclusion: The taste evaluation results show that the prednisone enteric-coated microsphere of the present disclosure has an excellent taste-masking effect, and exhibits a significantly-better taste than the commercially-available prednisone tablet.

### Example 8 Bioequivalency Test

Research description: The relative bioavailability and pharmacokinetic characteristics of 5 mg of the prednisone enteric-coated dry suspension (specification: 5 mg) in Example 1 and 5 mg of the commercially-available prednisone tablet as a reference preparation (trade name: Prednisone®; specification: 5 mg; West-Ward Pharmaceuticals International Ltd.) each orally administered once (fasting) to healthy humans in China were investigated, and the bioequivalency and the intra-individual variability of the two preparations orally administered to the healthy humans in China in a fasting state were preliminarily evaluated.

This research was a single-center, randomized, open, fasting single administration, two-preparation, two-sequence, two-cycle, and double-cross bioequivalency comparative study. This fasting study planned to enroll 10 subjects (including both males and females), and the subjects were administered in a double-cross order (two administration sequence groups T-R and R-T, the subjects were randomly assigned to one of the sequence groups, with 5 subjects in each sequence group). Determination of PK blood collection points: For each subject, blood sample collection must be conducted at 0 h before administration (within −60 min) and 0.25 h, 0.5 h, 0.75 h, 1 h, 1.5 h, 2 h, 2.5 h, 3 h, 3.5 h, 4 h, 4.5 h, 5 h, 6 h, 7 h, 8 h, 12 h, and 24 h (a total of 18 blood collection points) after administration in each cycle. All subjects were subjected to a fasting test. Results of statistical analysis for pharmacokinetics were as follows:

TABLE 7
Statistical results of pharmacokinetic data of prednisone in the fasting test

| Dependent | RefGeoLSM | TestGeoLSM | Ratio_% Ref_{—} | CI_90_Lower | CI_90_Upper | Power_80_20 |
|---|---|---|---|---|---|---|
| Ln(AUCINF_obs) | 129.55 | 132.39 | 102.19 | 95.59 | 109.26 | 1 |
| Ln(AUClast) | 123.99 | 122.93 | 99.14 | 93.65 | 104.95 | 1 |
| Ln(Cmax) | 25.46 | 24.22 | 95.11 | 88.13 | 102.64 | 1 |

TABLE 8
Statistical results of pharmacokinetic data of prednisolone in the fasting test

| Dependent | RefGeoLSM | TestGeoLSM | Ratio_% Ref_{—} | CI_90_Lower | CI_90_Upper | Power_80_20 |
|---|---|---|---|---|---|---|
| Ln(AUCINF_obs) | 635.12 | 639.21 | 100.64 | 95.05 | 106.57 | 1 |
| Ln(AUClast) | 608.94 | 597.88 | 98.18 | 93.86 | 102.71 | 1 |
| Ln(Cmax) | 161.84 | 123.85 | 76.53 | 72 | 81.34 | 1 |

In terms of AUC, the two preparations are within an equivalency range. The enteric-coated preparation has a slightly-smaller Cmax than the commercially-available prednisone tablet due to delayed dissolution, but this test shows that the two are still within an equivalency range. The enteric-coated preparation delays a peak time of prednisone, and exhibits the delayed dissolution characteristic and the enteric coating characteristic.

Because prednisone can be rapidly degraded and converted into prednisolone in the gastrointestinal tract, peak times of prednisone in the prednisone tablet and prednisolone both are short. It can be seen from test results that prednisolone undergoes faster absorption than prednisone, and has shorter Tmax than prednisone and significantly-larger Cmax and AUC than prednisone. Due to the delay of a peak time, the enteric-coated preparation has a great impact on Cmax of prednisolone. It is speculated that the enteric-coated preparation undergoes slower degradation in the intestinal tract than in the gastrointestinal tract. Therefore, AUC is within an equivalency range, but Cmax is slightly small and cannot reach an equivalency range, which is consistent with the Tmax delay. However, a pharmacological effect of this drug in vivo is not concentration-dependent. The AUC equivalency can be recognized as efficacy equivalency.

In summary, the Cmax and AUC of the prednisone enteric-coated preparation are in equivalency ranges, AUC of a metabolite is in an equivalency range, and Tmax of the metabolite is significantly delayed, which is in line with the characteristics of the enteric-coated preparation.

What is claimed is:

1. A prednisone enteric-coated preparation, comprising a prednisone micro-enteric-coated preparation unit as an active ingredient and a pharmaceutical adjuvant as an inactive ingredient.

2. The prednisone enteric-coated preparation according to claim 1, wherein the prednisone micro-enteric-coated preparation unit is selected from a prednisone enteric-coated microsphere, pellet, microcapsule, and microtablet, and the prednisone micro-enteric-coated preparation unit comprises 10% to 40% of prednisone and 60% to 90% of a pharmaceutical enteric polymer material in mass percentages; and the pharmaceutical enteric polymer material is selected from one or a combination of two or more of a methacrylic acid copolymer, cellulose acetate phthalate, hypromellose phthalate, hypromellose acetate succinate, and carboxy methyl ethyl cellulose.

3. The prednisone enteric-coated preparation according to claim 2, wherein the prednisone enteric-coated microsphere comprises 25% to 35% of the prednisone and 65% to 75% of the pharmaceutical enteric polymer material in mass percentages.

4. The prednisone enteric-coated preparation according to claim 3, wherein the prednisone enteric-coated microsphere comprises 30% of the prednisone and 70% of the pharmaceutical enteric polymer material in mass percentages.

5. The prednisone enteric-coated preparation according to claim 3, wherein the prednisone enteric-coated microsphere is prepared by one or a combination of two or more of a solvent evaporation method, an extrusion-spheronization method, a centrifugal granulation method, and a fluidized bed coating method, and the prednisone enteric-coated microsphere has an average particle size of 100 μm to 1,000 μm, an encapsulation rate of greater than 80%, a 2 h dissolution rate of less than 10% in an acid, and a 30 min dissolution rate of greater than 80% in a pH 6.8 buffer.

6. The prednisone enteric-coated preparation according to claim 1, wherein the prednisone enteric-coated preparation is an enteric-coated suspension, an enteric-coated granule, an enteric-coated capsule, an enteric-coated dispersible tablet, or an enteric-coated tablet.

7. The prednisone enteric-coated preparation according to claim 6, wherein the enteric-coated suspension comprises a prednisone enteric-coated microsphere as an active ingredient and a suspending agent, a corrigent, a filler, and a fragrance, and the suspending agent, the corrigent, the filler, and the fragrance are pharmaceutically-acceptable.

8. The prednisone enteric-coated preparation according to claim 7, wherein the suspending agent is selected from one or more of xanthan gum, sodium carboxymethyl cellulose, colloidal microcrystalline cellulose, hypromellose, gum arabic, and tragacanth, and a weight percentage of the suspending agent is 1.0% to 10.0%; and/or
the corrigent is selected from one or more of sucrose, aspartame, acesulfame potassium, sucralose, sodium saccharin, citric acid, and tartaric acid, and a weight percentage of the corrigent is 1.0% to 10.0%; and/or
the filler is selected from one or more of sucrose, corn starch, microcrystalline cellulose, lactose, and maltitol, and a weight percentage of the filler is 10.0% to 90.0%; and/or
the fragrance is selected from one or more of a peppermint fragrance, a peach fragrance, a strawberry fragrance, an orange fragrance, and a lemon fragrance, and a weight percentage of the fragrance is 0.1% to 5.0%.

9. The prednisone enteric-coated preparation according to claim 8, wherein types and weight percentage contents of the suspending agent, the corrigent, the filler, and the fragrance are as follows: 4.0% to 7.0% of the xanthan gum; 10.0% to 90.0% of the lactose; 1.0% to 2.0% of the aspartame; 1.0% to 2.0% of the acesulfame potassium; 2.0% to 5.0% of the citric acid; 0.1% to 0.5% of the peppermint fragrance; and 0.5% to 1.0% of the peach fragrance.

10. The prednisone enteric-coated preparation according to claim 9, wherein the prednisone enteric-coated preparation comprises the following components in weight percentages: 15.00% of the prednisone micro-enteric-coated preparation unit; 6.00% of the xanthan gum; 72.40% of the lactose; 1.50% of the aspartame; 1.00% of the acesulfame potassium; 3.00% of the citric acid; 0.30% of the peppermint fragrance; and 0.80% of the peach fragrance.

11. The prednisone enteric-coated preparation according to claim 6, wherein the enteric-coated capsule comprises a prednisone enteric-coated microsphere as an active ingredient and a filler as a pharmaceutically-acceptable adjuvant.

12. The prednisone enteric-coated preparation according to claim 11, wherein the filler is selected from one or more of sucrose, corn starch, microcrystalline cellulose, lactose, and maltitol, and a weight percentage of the filler is 10.0% to 90.0%.

13. The prednisone enteric-coated preparation according to claim 12, wherein a content in the enteric-coated capsule comprises the following components in weight percentages: 22% of the prednisone enteric-coated microsphere and 78% of granulated lactose; and a capsule shell of the enteric-coated capsule is gastric-soluble.

14. The prednisone enteric-coated preparation according to claim 6, wherein the enteric-coated dispersible tablet comprises a prednisone enteric-coated microsphere as an active ingredient and a filler, a disintegrant, a binder, and a lubricant, and the filler, the disintegrant, the binder, and the lubricant are pharmaceutically-acceptable.

15. The prednisone enteric-coated preparation according to claim 14, wherein the disintegrant is one or more of crospovidone, sodium carboxymethyl starch, croscarmellose sodium, microcrystalline cellulose, dry starch, and low-substituted hydroxypropyl cellulose, and a weight percentage of the disintegrant is 1.0% to 7.0%; and/or
the binder is one or more of povidone, hypromellose, hydroxypropyl cellulose, sodium carboxymethyl cellulose, and a starch slurry, and a weight percentage of the binder is 1.0% to 7.0%; and/or
the lubricant is one or more of magnesium stearate, talc, colloidal silicon dioxide, and stearic acid, and a weight percentage of the lubricant is 0.1% to 1.0%.

16. The prednisone enteric-coated preparation according to claim 15, wherein types and weight percentage contents of the filler, the disintegrant, the binder, and the lubricant are as follows: 10.0% to 40.0% of lactose; 10.0% to 50.0% of the microcrystalline cellulose; 2.0% to 5.0% of povidone K30; 2.0% to 5.0% of the croscarmellose sodium; 2.0% to 5.0% of the crospovidone; and 0.01% to 0.1% of the colloidal silicon dioxide.

17. The prednisone enteric-coated preparation according to claim 16, wherein the prednisone enteric-coated preparation comprises the following components in weight percentages: 10% of the prednisone enteric-coated microsphere; 35% of the lactose; 46.95% of the microcrystalline cellulose; 2% of the povidone K30; 3% of the croscarmellose sodium; 3% of the crospovidone; and 0.05% of the colloidal silicon dioxide.

18. A preparation method of the prednisone enteric-coated preparation according to claim 7, comprising: preparing the prednisone enteric-coated microsphere; mixing ½ of xanthan gum, lactose, aspartame, acesulfame potassium, and citric acid in a wet granulator, adding an appropriate amount of purified water to prepare a soft material, subjecting the soft material to fluidized bed drying and sieving to obtain a treated soft material; adding a remaining ½ of the xanthan gum, a peppermint fragrance, and a peach fragrance to the treated soft material, and mixing to obtain a mixture, and finally mixing the mixture with the prednisone enteric-coated microsphere thoroughly.

19. A preparation method of the prednisone enteric-coated preparation according to claim 11, comprising: preparing the prednisone enteric-coated microsphere; thoroughly mixing granulated lactose with the prednisone enteric-coated microsphere to obtain a mixture, and filling the mixture in a 5# gastric-soluble capsule shell to obtain a prednisone enteric-coated microsphere capsule.

20. A preparation method of the prednisone enteric-coated preparation according to claim 14, comprising: preparing the prednisone enteric-coated microsphere; mixing lactose, microcrystalline cellulose, and povidone K30 in a wet granulator, adding an appropriate amount of purified water to prepare a soft material, subjecting the soft material to fluidized bed drying and sieving to obtain a treated soft material; adding croscarmellose sodium, crospovidone, and colloidal silicon dioxide to the treated soft material, and thoroughly mixing to obtain a first mixture, and finally mixing the first mixture with the prednisone enteric-coated microsphere thoroughly to obtain a second mixture, and obtaining a prednisone enteric-coated dispersible tablet by tableting the second mixture with a rotary tablet press.

21. The preparation method according to claim 18, wherein a preparation method of the prednisone enteric-coated microsphere comprises: dissolving a pharmaceutical enteric polymer material in an organic solvent, adding a prednisone raw material to produce an oil phase, adding the oil phase to an aqueous phase containing an emulsifier to obtain a mixture, stirring the mixture to produce an emulsion droplet, and solidifying the emulsion droplet to obtain the prednisone enteric-coated microsphere.

22. The preparation method according to claim 21, wherein in the preparation method of the prednisone enteric-coated microsphere, the organic solvent is selected from one or more of methyl acetate, ethyl acetate, dichloromethane, ethanol, and n-heptane; and/or the emulsifier is selected from one or more of Tween, Span, sodium dodecyl sulfate, and polyvinyl alcohol.

23. The prednisone enteric-coated preparation according to claim 1, wherein the prednisone enteric-coated preparation is used for an allergic disease, an autoimmune inflammatory disease, acute leukemia, or malignant lymphoma; and the allergic disease is a connective tissue disease, systemic lupus erythematosus, severe polymyositis, severe bronchial asthma, dermatomyositis, or vasculitis.

24. The prednisone enteric-coated preparation according to claim 1, wherein the prednisone enteric-coated preparation is used for a patient with difficult swallowing.

25. A preparation method of a pharmaceutical preparation, comprising using the prednisone enteric-coated preparation according to claim 1, wherein the pharmaceutical preparation is used for an allergic disease, an autoimmune inflammatory disease, acute leukemia, or malignant lymphoma, wherein the allergic disease is a connective tissue disease, systemic lupus erythematosus, severe polymyositis, severe bronchial asthma, dermatomyositis, or vasculitis.

26. A preparation method of a pharmaceutical preparation, comprising using the prednisone enteric-coated preparation according to claim 1, wherein the pharmaceutical preparation is used for a patient with difficult swallowing.

27. A method for treating an allergic disease, an autoimmune inflammatory disease, acute leukemia, or malignant lymphoma, comprising administering the prednisone enteric-coated preparation according to claim 1 to a patient, wherein the allergic disease is a connective tissue disease, systemic lupus erythematosus, severe polymyositis, severe bronchial asthma, dermatomyositis, or vasculitis.
